(19) United States
(12) Patent Application Publication
(10) Pub. No.: US 2003/0176508 A1
(43) Pub. Date: Sep. 18, 2003

(54) Ferulic acid ethyl-guaiacol or vinyl-guaiacol as preservative combination in cosmetic compositions

(76) Inventors: Peter Samuel James Cheetham, Tunbridge Wells (GB); Michelle Lorraine Gradley, Canterbury (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/344,397

(22) PCT Filed: Aug. 10, 2001
(86) PCT No.: PCT/GB01/03602

(30) Foreign Application Priority Data
Aug. 11, 2000 (GB) 0019768.1

(51) Int. Cl. A61K 31/19; A61K 31/05

(52) U.S. Cl. 514/570; 514/731

(57) ABSTRACT

A composition of ferulic acid and ethylguaiacol or vinylguaiacol (preferably as the free compounds, optionally as salts or derivatives) shows synergistic antimicrobial properties and may be used in personal care products.

### TECHNICAL FIELD

The present invention relates to compositions having antimicrobial (ie, antibacterial and/or antifungal) activity, and to their use in personal care compositions.

The demand for natural preservative agents is great and increasing in many areas of industry including food, cosmetics, hygiene and personal care. However, this demand is either unmet or met with agents which are unsatisfactory. For example, synthetic chemical preservatives may have adverse environmental effects as they tend to be poorly biodegradable or liberate undesirable compounds such as formaldehyde.

### BACKGROUND ART

Our related application, WO 00/47045, filed Feb. 14, 2000, discloses that a broad class of compounds, including vinylguaiacol and ethylguaiacol, can be used as multifunctional preservatives. Among the activities of the preservatives is antimicrobial activity.

GB-A-2289676 concerns the use of ferulic acid for retaining food freshness. Other antimicrobial and antioxidant components may be included. WO 98/02044 discloses disinfectants that may include ferulic acid.

U.S. Pat. No. 6,114,377 discloses compositions which may include ferulic acid as an anti-irritant.

### DISCLOSURE OF INVENTION

The present invention is based on the surprising finding that combinations of specific compounds have greater antimicrobial activity than corresponding amounts of the individual compounds used alone. Therefore, the present invention permits lower amounts of the compounds to be used and/or provides a higher antimicrobial activity for a given amount of composition.

Accordingly, the present invention provides a composition comprising (a) a first compound which is ferulic acid and/or a salt and/or derivative thereof, and (b) at least one further compound selected from ethylguaiacol, vinylguaiacol and salts and/or derivatives thereof.

In another aspect, the present invention provides the use of such a composition as an antimicrobial agent.

This invention involves the use of two or more compounds (preferably two compounds) as an antimicrobial agent. The compounds are selected from ferulic acid (ie, 4-hydroxy-3-methoxycinnamic acid), vinylguaiacol (ie, 4-hydroxy-3-methoxystyrene) and ethylguaiacol (ie, 4-ethyl-2-methoxyphenol) and their salts and derivatives.

Preferably, the compounds are used in their free, underivatised form. However, salts and/or derivatives of the compounds may also be used in the invention.

Suitable salts include acid salts formed by deprotonation of a carboxylic acid and/or phenolic hydroxyl group. Typically, the salts comprise metal or ammonium cations. The metal cations may be mono-, di- or tri-valent. Preferred metal cations are alkali metals such as sodium and potassium, for example. Ammonium cations include, for example, cations of formula R¹R²R³R⁴N⁺, wherein R¹, R², R³ and R⁴ are independently selected from H, alkyl, alkaryl and aryl.

Derivatives of the compounds are any derivatives which do not affect the activity of the compounds as antimicrobial agents. Preferred derivatives include esters of ferulic acid with alcohols and phenols, especially those which are readily hydrolysable. Alcohols and phenols include, for example, compounds of formula R⁵OH, where R⁵ is alkyl, alkaryl or aryl. Also suitable are amides of ferulic acid, obtainable by reaction with amines of formula R⁶R⁷R⁸NH, wherein R⁶, R⁷ and R⁸ are independently selected from H, alkyl, alkaryl and aryl. Other suitable derivatives include esters obtainable by reaction of the phenolic hydroxyl group in the compounds with a carboxylic acid of formula R⁹COOH, wherein R⁹ is H, alkyl, alkaryl or aryl, especially where the resulting ester is readily hydrolysable.

The term “alkyl”, as used herein, includes straight or branched chain C₁ to C₁₂, preferably C₁ to C₆ groups (eg, methyl, ethyl and propyl) and, for C₃ to C₁₂, includes cycloalkyl (eg, cyclohexyl). Alkaryl means alkyl substituted with alkyl eg, benzyl. Aryl includes carbocyclic and heterocyclic aromatic ring systems (eg, phenyl and naphthyl) which can optionally be substituted on the aromatic ring with one or more groups (eg, alkyl, OH or COOH).

Preferred compositions according to the invention are those comprising: ferulic acid and ethylguaiacol; or ferulic acid and vinylguaiacol. These have particularly effective antibacterial activity.

It is preferred that, in the compositions of the present invention, the ratio of each compound to each other compound, by weight, is in the range of from 10:1 to 1:10, more preferably 5:1 to 1:5, most preferably 2:1 to 1:2, even more preferably 1.5:1 to 1:1.5.

The compositions of the invention may have a range of different pH values. For example, certain compositions of the invention may have a pH around neutral ie, from 6 to 8. Other compositions of the invention may have a pH of less than 7 (such as, for example, a pH of from 4 to 6).

The present invention can be applied in many different technical areas. Preferably, the compositions are used in the personal care, dermatological or household product areas. The compositions of the invention can, therefore, be personal care compositions, dermatological compositions or household product compositions. Such compositions may comprise a surfactant, for example in an amount of from 0.01 to 50% (preferably 1 to 30%) by weight. Preferred personal care compositions include: lotions or creams for application to the skin; cosmetics; hair care or body care compositions such as shampoos (including antidandruff shampoos) and cleansing compositions. Particularly preferred compositions according to the invention are hand lotions or shampoos. These compositions will typically include one or more other components, such as, for example, fragrance, colourants, UV absorbers, a-hydroxy acids, antioxidants and other components which are active ingredients and/or cosmetically acceptable diluents and/or carriers.

Compositions of the invention may, therefore, be finished products (ie, intended for direct use by the consumer) in the intended area (eg, personal care compositions). In these compositions, the total amount of the compounds in the compositions preferably ranges from 0.01 to 5.0% by weight, more preferably 0.05 to 4.0% by weight, most preferably 1.0 to 4.0% by weight (such as 2.0 to 3.0% by weight).

The compositions of the invention can, alternatively, be suitable for use as antimicrobial agents for addition to a product. In this case, the amount of the compounds in the composition will preferably be relatively high (eg, from 50 to 100% by weight of the composition). Compositions for use as antimicrobial compositions may contain additives such as, for example, other compounds known to have antimicrobial activity, and carriers and/or diluents (eg, alcoholic and/or aqueous solvents).

The compositions of the invention may be effective against a range of microorganisms. For example microorganisms against which the compositions of the invention may be used include Bacillus subtilis, Enterobacter aerogenes, Escherichia coli, Klebsiella pneumoniae, Proteus vulgaris, Pseudomonas aeruginosa, Pseudomonas aeruginosa, Salmonella choleraesuis, Staphylococcus aureus, Aspergillus niger, Saccharomyces cerevisiae, Pityosporium ovale, Staphylococcus epidermidis, Propionobacter acnes and Corynebacterium xerosis.

### MODES FOR CARRYING OUT THE INVENTION

The invention will now be described with reference to the following non-limiting examples. In the examples and throughout the specification, all percentages are by weight unless otherwise indicated.

### EXAMPLES

### Materials and Methods

Hand Lotion and Shampoo Test

Hand lotion and shampoo compositions of the invention were prepared according to the formulations listed in Tables A and B, with the compositions of Tables A and B being weighed out in 25-gram aliquots in one-ounce (30 g) glass jars and preserved with one or more additions of 0.25 ml of a 10% solution of the compounds (ie, ferulic acid (FA), vinylguaiacol (VG) and/or ethylguaiacol (EG)) in methanol to give an overall concentration in the product of 1000 ppm.

TABLE A
Shampoo Formulation

| Ingredient | % | Actual Amount (g) |
|---|---|---|
| D.I. Water | 64.5 | 1612.50 |
| Ammonium Laureth Sulfate | 30 | 750.00 |
| Hydrolyzed Animal | 2 | 50.03 |
| Protein | | |
| Cocamide DEA | 2 | 54.25 |
| Cocamidopropyl Betaine | 1 | 25.07 |
| Sodium Chloride | 0.5 | 12.51 |
| Total | 100 | 2504.36 |

Shampoo Procedure

All ingredients were weighed and added to a 4000 mL beaker in the order listed. They were mixed with a motorized paddle-type propeller stirrer (Caframo model RZR 50) at 200 rpm at room temperature for approximately two hours. The shampoo did not appear thoroughly mixed so stirring was continued at 800 rpm for an additional 30 minutes.

TABLE B
Hand Lotion Formulation

| Phase | Ingredient | % |
|---|---|---|
| A | Deionized Water | 84.16 |
| A | Carbomer: Carbopol 934 | 0.08 |
| B | Propylene Glycol | 5.00 |
| C | Cetyl Alcohol | 0.75 |
| C | Stearic Acid | 3.00 |
| C | Stearyl Alcohol | 0.38 |
| C | Glyceryl Monostearate | 1.00 |
| | S.E. | |
| C | Mineral Oil | 3.00 |
| C | Silicone | 0.50 |
| D | Deionized Water | 2.00 |
| D | Triethanolamine | 0.13 |

Hand Lotion Procedure

Phase B, C, and D are prepared by weighing the ingredients into separate beakers. Phase A is prepared by slowly sprinkling the Carbomer into the vortex of the stirring water. When the Carbomer is dissolved, begin heating and continue agitation. Heat phase B to 80° C. and add to Phase A when A has reached 60° C. Continue to heat and agitate water phase until it reaches 75° C. Heat Phase C to 75° C. and slowly add to water phase when both are at 75 to 80° C. Continue agitation. Add Phase D to the emulsion when it has cooled to 60° C. Agitate on medium speed until the lotion cools to 40° C.

The samples were tested by the challenge test described below. In this challenge test, samples were stored at room temperatures, inoculated at zero days and streaked at 0 days, 1 day, 2 days, 1 week, 2 weeks and 3 weeks.

The data for the tests of hand lotion samples preserved with ferulic acid, ethylguaiacol and vinylguaiacol are listed in Tables 1 and 2.

The combinations of 1000 ppm FA & 1000 ppm EG and 1000 ppm FA & 1000 ppm VG were most effective against bacteria, controlling growth within one day as highlighted in Table 1. The combination of 1000 ppm EG & 1000 ppm VG was most effective against the fungi, also controlling growth within one day.

TABLE 1
Test of Hand Lotion With Bacteria Pool

| ppm | | | Bacterial Growth Rating | | | | |
|---|---|---|---|---|---|---|---|
| ferulic | ppm | ppm | 1 | 2 | 1 | 2 | 3 |
| acid | vinylguaiacol | ethylguaiacol | day | day | wk | wk | wk |
| 0 | 0 | 1000 | 9 | 6 | 5 | 4 | 1 |
| 0 | 1000 | 0 | 4 | 3 | 4 | 3 | 1 |
| 1000 | 0 | 0 | 7 | 6 | 9 | 6 | 9 |
| 0 | 1000 | 1000 | 2 | 3 | 3 | 1 | 1 |
| 1000 | 0 | 1000 | 1 | 1 | 1 | 1 | 1 |
| 1000 | 1000 | 0 | 1 | 1 | 1 | 1 | 1 |

TABLE 2
Test of Hand Lotion With A. niger and S. cerevisiae

| ppm | | | Bacterial Growth Rating | | | | |
|---|---|---|---|---|---|---|---|
| ferulic | ppm | ppm | 1 | 2 | 1 | 2 | 3 |
| acid | vinylguaiacol | ethylguaiacol | day | day | wk | wk | wk |
| 0 | 0 | 1000 | 4/4 | 3/4 | 3/4 | 2/2 | 3/3 |
| 0 | 1000 | 0 | 3/2 | 1/3 | 1/2 | 1 | 1 |
| 1000 | 0 | 0 | 4/6 | 4/6 | 3/3 | 4/3 | 3/4 |
| 0 | 1000 | 1000 | 1 | 1 | 1 | 1 | 1 |
| 1000 | 0 | 1000 | 3/2 | 2/2 | 1 | 1/2 | 1 |
| 1000 | 1000 | 0 | 2/3 | 1 | 1/2 | 1 | 1 |

TABLE 3
Test of Shampoo With Bacteria Pool

| ppm | | | Bacterial Growth Rating | | | | |
|---|---|---|---|---|---|---|---|
| ferulic | ppm | ppm | 1 | 2 | 1 | 2 | 3 |
| acid | vinylguaiacol | ethylguaiacol | day | day | wk | wk | wk |
| 0 | 0 | 1000 | 9 | 9 | 9 | 9 | 9 |
| 0 | 1000 | 0 | 9 | 9 | 8 | 9 | 9 |
| 1000 | 0 | 0 | 9 | 9 | 9 | 9 | 9 |
| 0 | 1000 | 1000 | 9 | 9 | 3 | 1 | 1 |
| 1000 | 0 | 1000 | 9 | 9 | 1 | 1 | 3 |
| 1000 | 1000 | 0 | 9 | 9 | 2 | 1 | 1 |

Challenge Test Procedure with Bacterial Pool

1. Twenty-five grams of sample are weighed out in one-ounce glass jars.

2. Biocide was dissolved into solution before use and different concentrations are prepared by adding the appropriate amount of the biocide solution to the samples.

3. A negative control is included using a sample without any biocide.

4. The bacteria pool inoculum is made fresh on the day of inoculation by using equal volumes of the 24 hour cultures of the ATCC bacteria grown individually in nutrient broth at 30° C. The bacteria pool consists of the following:

| | | |
|---|---|---|
| Bs | Bacillus subtilis ATCC** | #8473 |
| Ea | Enterobacter aerogenes | #13048 |
| Ec | Escherichia coli | #11229 |
| Kp | Klebsiella pneumoniae | #8308 |
| Pv | Proteus vulgaris | #881 |
| PRD10 | Pseudomonas aeruginosa | #15442 |
| Pa | Pseudomonas aeruginosa | #10145 |
| Sc | Salmonella choleraesuis | #10708 |
| Sa | Staphylococcus aureus | #6538 |

5. Before inoculation, samples are streaked on TSA (tryptic soy agar) plates with sterile cotton swabs. The plates are incubated at 30° C. for 48 hours and then read using a bacterial colony counter and rated for microbial growth according to the Growth Rating Chart.

Growth Rating Chart

| rating | number of colonies |
|---|---|
| 1 | 0 (No Growth) |
| 2 | 1-4 |
| 3 | 5-10 |
| 4 | 11-25 |
| 5 | 26-50 |
| 6 | 51-100 |
| 7 | 101-200 |
| 8 | 201-300 |
| 9 | (TNTC) too numerous to count |
| 10 | (TNTC) solid m |

6. Samples are inoculated with 0.05 mL (for 25 g samples) of the 9 pool inoculum.

7. Immediately after inoculation, samples are streaked with sterile cotton swabs on TSA plates. The plates are then incubated at 30° C. for 48 hours. The samples are incubated at room temperature.

8. Samples are streaked again at 1 day, 2 days, 1 week, 2 weeks and 3 weeks.

9. All plates are incubated 48 hours at 30° C. then read and rated according to the Growth Rating Chart.

10. Samples are kept at room temperature.

Challenge Test Procedure with Aspergillus niger and Saccharomyces cerevisiae

1. Twenty-five grams of sample are weighed out in one-ounce glass jars.

2. Biocide is dissolved into solution before use and different concentrations are prepared by adding the appropriate amount of the biocide solution to the samples.

3. A negative control is included using a sample without any biocide.

4. Before inoculation, samples are streaked on MYA (malt yeast agar) plates with sterile cotton swabs. The plates are incubated at 30° C. for 48 hours and then read using a bacterial colony counter and rated for microbial growth according to the Growth Rating Chart.

Growth Rating Chart

| rating | number of colonies |
|---|---|
| 1 | 0 (No Growth) |
| 2 | 1-4 |
| 3 | 5-10 |
| 4 | 11-25 |
| 5 | 26-50 |
| 6 | 51-100 |
| 7 | 101-200 |
| 8 | 201-300 |
| 9 | (TNTC) too numerous to count |
| 10 | (TNTC) solid m |

5. The inoculum is prepared by adding 9.9 mL sterile 0.85% saline solution to a pure culture slant grown on MYA. A cotton-tipped swab is used to gently rub the surface of the slant to free the cells. The resulting suspension is diluted by transferring 1 mL of the suspension to 9 mL sterile saline. The yeast and fungus used are:

| | | |
|---|---|---|
| An | Aspergillus niger ATCC** | #16404 |
| Sc | Saccharomyces cerevisiae | #4105 |

6. Samples are inoculated with 0.05 mL (for 25 g samples) of the An suspension and 0.05 mL of the Sc suspension.

7. Immediately after inoculation, samples are streaked with sterile cotton swabs on MYA plates. The plates are then incubated at 30° C. for 48 hours. The samples are incubated at room temperature.

8. Samples are streaked again at 1 day, 2 days, 1 week, 2 weeks and 3 weeks.

9. All plates are incubated 48 hours at 30° C. then read and rated according to the Growth Rating Chart. Samples are kept at room temperature.

1. Composition comprising (a) a first compound which is ferulic acid and/or a salt and/or derivative thereof, and (b) at least one further compound selected from ethylguaiacol, vinylguaiacol and salts and/or derivatives thereof.

2. Composition as claimed in claim 1 which has antimicrobial activity.

3. Composition as claimed in claim 1 or claim 2 comprising ferulic acid and ethylguaiacol.

4. Composition as claimed in claim 1 or claim 2 comprising ferulic acid and vinylguaiacol.

5. Composition as claimed in claim 3 or claim 4 which has antibacterial activity.

6. Composition as claimed in any one of claims 1 to 5 wherein a compound (a) and a compound (b) are present in a ratio by weight of from 10:1 to 1:10.

7. Composition as claimed in any one of claims 1 to 5, wherein a compound (a) and a compound (b) are present in a ratio by weight of from 5:1 to 1:5.

8. Composition as claimed in any one of claims 1 to 5, wherein a compound (a) and a compound (b) are present in a ratio by weight of from 2:1 to 1:2.

9. Composition as claimed in any one of claims 1 to 8 which has a pH of from 6 to 8.

10. Composition as claimed in any one of claims 1 to 8 which has a pH of less than 7.

11. Composition as claimed in any one of claims 1 to 8 which has a pH of from 4 to 6.

12. Composition as claimed in any one of claims 1 to 11 which is a personal care or cosmetic composition.

13. Composition as claimed in claim 12 which is a hand lotion or shampoo.

14. Composition as claimed in any one of claims 1 to 13, wherein the total amount of the ferulic acid, salt and/or derivative thereof and the at least one further compound is from 0.01 to 5.0% by weight of the composition.

15. Use of a composition comprising ferulic acid and/or a salt and/or derivative thereof, and at least one further compound selected from ethylguaiacol, vinylguaiacol and salts and/or derivatives thereof as an antimicrobial agent.

16. Use according to claim 15 wherein the composition is as defined in any of claims 3 to 14.
